(19) United States
(12) Patent Application Publication
(10) Pub. No.: US 2006/0073098 A1
(43) Pub. Date: Apr. 6, 2006

(54) Irrigating solution for neurosurgical procedures

(76) Inventor: Yanming Wang, Malden, MA (US)

Correspondence Address: YANMING WANG, 203 SUMMER STREET, MALDEN, MA, 02148, US

(21) Appl. No.: 11/243,875

(22) Filed: Oct. 5, 2005

Related U.S. Application Data
(63) Continuation of application No. 10/956,453, filed on Oct. 2, 2004.
(63) Continuation of application No. 11/193,181, filed on Jul. 28, 2005.

(51) Int. Cl. A61K 38/28; A61K 49/00; A61K 31/70

(52) U.S. Cl. 424/9.1; 514/23; 514/3; 514/2; 514/47; 424/602

(57) ABSTRACT

Saline is harmful to neuronal tissue as an irrigating solution during neurosurgical procedures. An irrigating solution to replace the use of saline during surgical procedure is provided. The irrigating solution includes polypeptides, magnesium, insulin and ATP in artificial CSF.

This is a continuation of the patent application filed Oct. 2, 2004, application Ser. No. 10/956,453; and the patent application filed Jul. 28, 2005, application Ser. No. 11/193,181. This invention is related to a lymph-like fluid composition for irrigating and protecting brain and spinal cord during neurosurgical procedures.

### BACKGROUND OF THE INVENTION

1. Field of the Invention

2. Background Information

Neurosurgical procedures routinely require that application of fairly copious amounts of liquid to irrigate and protect operating field. Neurosurgeons are still using physiological saline to irrigate. In lengthy open neurosurgical procedures, it is quite possible that copious use of saline as an irrigant can completely replace the cerebrospinal fluid (CSF). Physiological saline is 0.9% sodium chloride, although it is isotonic, it is not ‘physiological’ at all. Saline has been reportedly harmful as an irrigant during neurosurgical procedure (Uchida K et al. Possible harmful effects on central nervous system cells in the use of physiologyical saline as an irrigant during neurosurgical procedures. Surg Neurol 2004; 62:96-105). Elliot B solution is an artificial CSF that has been approved as a solvent for intrathecal administration of drug since 1996 in USA. Although the CSF consists of more integrated electrolytes than saline, recent study has shown that the CSF also mediates central nervous system (CNS) injury.

The central nervous system (CNS) including brain and spinal cord is extremely susceptible to hypoxic-ischemic insults compared with peripheral organ systems such as the liver, kidney, lung, or intestines. It has been shown that the existence of the CSF in the CNS may be one of the important mechanisms underlying this susceptibility.

In peripheral tissues, capillaries are relatively permeable and as a result the interstitial fluid (ISF) contains about 2 g/dl of plasma proteins. It is believed that interstitial proteins and hyaluronic acids form a dense network of proteoglycan filaments so that the ISF moves molecule by molecule from one place to another by kinetic motion among pro-teoglycan filaments in the interstitium. Normally the amount of free-flowing fluid, present in the interstitium is small. A low interstitial protein concentration results in an increased amount of free ISF. An elevated concentration of interstitial protein may reduce Is the free ISF, but it also attracts more fluid, resulting in increased volume. The lymphatic system is the scavenging pathway for interstitial proteins. By regulating the removal of excess protein, the lymphatic system keeps the interstitial protein concentration around 2 g/dl. This ensures limited free fluid and also regulates the ISF volume. Lymph flow reduces ISF volume resulting in negative interstitial pressure. Therefore, the movement of proteins from plasma to ISF and finally to lymph is important for maintaining extracellular homeostasis.

The CNS lacks a lymphatic system; instead it is bathed by the CSF. The CSF is very different from the lymph in peripheral tissues in at least two aspects: protein concentration and the resultant interstitial fluid pressure. The CSF is secreted by the choroid plexuses that line the cerebral ventricles. Tight junctions linking the adjacent choroidal epithelial cells form the blood-CSF barrier and prevent most large molecules from passing into the CSF from the blood. Therefore the CSF contains an extremely low protein concentration. In a human adult, the CSF occupies about 10 percent of the intra-cranial and intra-spinal volume. The average rate of CSF formation is about 21 to 22 ml/hr, or approximately 500 ml/day. The choroid plexuses may not be the only sites for CSF production. Milhorat reported that in monkeys with choroid plexuses removed, up to 60% of the CSF is produced from ISF flow out of the brain. The blood-brain barrier (BBB) prevents proteins from entering the interstitium. Therefore, it is speculated that the ISF in brain, just like the CSF, has a low protein concentration. Importantly, the CSF is contiguous with the ISF, with the Virchow-Robin spaces, serving as a conduit. It is estimated that intracellular protein concentration averages about 16 g/dl in mammalian cells. Therefore water and Na⁺ in the ISF tend to move easily into cells. To make matters worse, the ICP averages about 10 mmHg leading to a positive interstitial fluid pressure. Taken together, these factors make the CNS prone to edema formation. As a result cells in the CNS constantly consume more energy to remove excess intracellular fluid in physiological condition. When cell energy is compromised, such as in ischemia, cells rapidly become swollen, i.e. cytotoxic edema.

Cerebral edema is a common pathological process to all CNS injuries. Brain and spinal cord manipulating, cutting, electrical coagulating, sucking, pressing, etc. during neurosurgical procedures, usually result in damage in local operating region. Therefore, cerebral edema following neurosurgical procedure is always inevitable. The treatment of many neurological diseases requires the surgical operation, such as head trauma, spinal cord trauma, cerebral aneurysm, brain tumor etc. Swelling of cerebral tissue can compress blood vessels inside the Virchow-Robin space leading to a persistent deficit in blood perfusion even after the restoration of blood perfusion, termed a ‘no-reflow’ or ‘low reflow’ phenomenon. This blood perfusion deficit blocks collateral circulation and induces a feed-back loop contributing irreversible cerebral cell death and tissue necrosis.

This invention discloses a lymph-like fluid composition for replacing the use of saline as an irrigating solution during neurosurgical procedures. The lymph-like fluid composition is based on mimicking lymph in peripheral organ system by adding polypeptides in artificial CSF.

Researchers have suggested that bolus infusion of hyperoncotic solution into the cerebral vasculature or perfusion of hyperoncotic artificial CSF can alleviate cerebral edema. The term “hyperonconic” refers to high colloid osmotic pressure caused by the existence of large molecular weight substances that do not pass readily across capillary walls. For example, U.S. Pat. No. 6,500,809 to Frazer Glenn discloses a method of treating neural tissue edema using hyperoncotic artificial CSF. Several colloid osmotic agents including albumin and dextran were used in the method.

This invention, however, reveals that the colloid osmotic pressure is not a key factor. Although albumin is effective in protecting the CNS tissue, it appears that its colloid osmotic effect is not the primary reason for its neural protective effect, because other colloid osmotic agents such as Dextran and Hetastarch are ineffective. In contrast, gelatins, even with molecular weights smaller than cut-off size for colloid osmotic agents are effective. In fact, gelatins with various molecular weights ranging from 20,000 to 100,000 Daltons are all effective regardless of their molecular weights. Collagen and Sericin peptides are also effective. Albumin, gelatin, collagen, and Sericin peptides all belong to poly amino acids category. It is thus the water and ions binding properties of proteins or other polyaminoacids that really matter.

This lymph-like fluid composition also directed at other mechanisms of ischemic injury that are common to all organ systems, including the use of insulin, magnesium and ATP.

Glucose is the major energy source for CNS. Neuronal cells are well known to require large amounts of glucose. The CSF contains about two third of plasma glucose concentration (CSF: 61 mg/dl; Plasma: 92 mg /dl). However it contains about at most one fifteenth of plasma insulin concentration (CSF: 0-4 μU/ml; fasting plasma: 20-30 μU/ml). Insulin is a polypeptide, with a molecular weight of about 6000 Daltons. Similar to albumin, it cannot easily enter the CSF through the blood-CSF barrier. Insulin has also been regarded as a growth factor, evidences have repeatedly proven that insulin yield protection for ischemic cerebral tissue independent of its glucose lowering effect. Compared with other growth factors, insulin has been used in clinic for years, and is much less expensive.

Magnesium (Mg²⁺) is the second highest electrolyte intracellularly (58 mEq/L). ATP (Adenosine 5′-triphosphate) is always present as a magnesium/ATP complex. Mg²⁺ basically provides stability to ATP. At least more than 260 to 300 enzymes have been found to require Mg²⁺ for activation. Best known among these are the enzymes involved in phosphorylations and dephosphorylations: ATPases, phosphatases, and kinases for glycolytic pathway and krebs cycles. At the level of the cell membrane Mg²⁺ is needed for cytoskeletal integrity, the insertion of protein into membranes, the maintenance of bilayer fluidity, binding of intracellular messengers to the membrane, regulation of intracellular Ca²⁺ release by inositol triphosphate etc. Mg²⁺ also affects the activities of pumps and channels regulating ion traffic across the cell membrane. The potential changes in tissue Mg²⁺ might also affect the tissue ATP levels. In tissue culture and animal models elevated Mg²⁺ concentration has been repeatedly proven to protect neurons and other cells.

The concentration of ATP inside cells is high, whereas the concentration outside cells is very low. Harkness and coworkers showed that the ATP concentrations is about 1 to 20 μ mol/l in plasma, however in CSF, ATP could not be detected, and it was estimated to be about less than 0.05 μ mol/l. Muñoz and coworkers detected that the ATP concentration in CSF is about 16 nM/l. Exogenous ATP provides direct energy to the damaged tissue. Sakama and coworkers showed that continuous application of ATP (100 μM) significantly increased axonal transport of membrane-bound organelles in anterograde and retrograde directions in cultured neurons. Uridine 5′-triphosphate produced an effect similar to ATP. Mg-ATP has been used clinically in Japan to treat hepatic and kidney hypoxia-ischemia.

Acidosis is a universal response of tissue to ischemia. In the brain, severe acidosis has been linked to worsening of cerebral infarction. Recent evidence however suggests that mild extracellular acidosis protects the brain probably through preventing activation of NMDA receptors and inhibition of Na⁺/H⁺ exchange. It has been reported mild acidosis provide cell protection down to pH 6.2. The acidosis that accompanies ischemia is an important endogenous protective mechanism. Correction of acidosis seems to trigger the injury. It has also been speculated that mild acidosis might stimulate anaerobic glycolysis that might supplement NADH oxidation and ATP yields.

### DETAILED DESCRIPTION OF AN ILLUSTRATIVE EMBODIMENT

The embodiment of the present invention introduces a lymph-like fluid composition for replacing the use of saline as an irrigating solution during neurosurgical procedures. The example components of the lymph-like fluid composition include (1) Molecules primarily consisting of chemically-linked amino acids, (2) ionic magnesium (Mg²⁺), (3) adenosine triphosphate (ATP), (4) insulin, and (5) artificial CSF.

### The Polypeptides

Acting as his own lexicographer, the patentee calls the molecules that mainly consist of chemically-linked amino acids as “the Polypeptides” for the sake of simplicity. The Polypeptides have significant water and ions binding capacity. They include a wide variety of molecules, from small peptides containing two or more amino acids to proteins of large molecular weight and multiple peptide chains. The Polypeptides can be natural or synthetic molecules. They also include molecules that consist of amino acids and other building blocks such as hyaluronic acid or glucose (e.g., proteoglycan). The polypeptides are used here to simulate the function of intestinal proteins.

Whether the Polypeptides can pass through the capillary walls to generate colloid osmotic pressure are not important in this invention. In fact, colloid osmotic agents with-out the Polypeptides, such as Dextran, do not confer neuroprotective effect. It is preferred that the Polypeptides do not readily pass through cell membrane. Therefore, the invention prefers, but is not limited to, Polypeptides with molecular weight between 1,000 to 30,000 Daltons.

Several examples the Polypeptides are described here, including albumin, collagen, gelatin and sericin. Albumin is plasma protein and an expensive option for the treatment, considering the current cost of albumin use already accounts for 10 to 30% of pharmacy budgets in hospital units.

Gelatins, on the other hand, can be a much cheaper option for the Polypeptides. Injectable gelatin polypeptides are much cheaper than albumin, and have been used in clinic in many countries such as Europe, China and South Africa. Examples of available commercial pharmaceutical gelatins include GELOFUSINE® and HAEMACCEL®. Is Sericin and Fibroin, the constituents from the silkworm cocoon, can also be a cheaper option for polypeptides. Examples of available commercial Sericin products are from Silk Biochemical Co Ltd (46-3-108, Zhao hui Yi Qu, Hangzhou, China), and Sinosilk Co Ltd (1 Jincheng Road, Wuxi, Jiangsu China). Various Silk peptides with molecular weight ranging from 300-100,000 can be obtained and be used as polypeptides. Heat shock protein can also be used as the Polypeptide. Example concentrations of the Polypeptides are ranged from 0.1-30 gram per dl. The preferred concentration range is between 1 and 10 gram per dl.

Insulin, ATP, and Other Constituents

The insulin concentration should be in a range from 0.01 to 1000 μU/ml. The preferred insulin concentration is between 1 and 60 μU/ml. All growth factors having insulin-like effect can be chosen to replace insulin. For examples, insulin-like growth factors, nerve growth factor, brain derived neurotrophic factor, neurotrophin, fibroblast growth factor and glial cell line derived neurotrophic factor, erythroproietin, growth hormone, and growth hormone releasing factor may be used to replace insulin or may be used in combination with insulin.

The ATP concentration should be in a range from 16 nM to 5 mM. The preferred ATP concentration is between 0.001 to 1 mM. The most preferred ATP concentration is between 0.001 and 0.01 mM. Other high energy compound such as Uridine 5′-triphosphate can be used to replace ATP.

The components and concentration range of the Mg²⁺ and artificial CSF can be as follow: Na 120-155 meq/L, K 0.1-5.0 meq/L, Ca 0.1-3.0 meq/L, P 0.1-2 meq/L, Cl 120-155 meq/L, Mg 0.4-8 meq/L, HCO₃ 0-25 meq/L, Glucose 0-60 mg/dl and water. The preferred concentration range of the Mg²⁺ and artificial CSF is as follow: Na 150 meq/L, K 3.0 meq/L, Ca 1.4 meq/L, P 1.0 meq/L, Cl 155 meq/L, Mg 2.5-5 meq/L, and water. Alternatively, Patient's own CSF may be used to replace artificial. CSF. Usually up to 160 ml of the patient's own CSF can be obtained each time. Elliot B solution is an artificial CSF that has been approved as a solvent since 1996 in USA.

Normal blood pH value is about 7.35 to 7.45. The pH value of the lymph-like fluid composition should be in a range between 6.2 to 7.45. The pH value between 6.8-7.0 is preferred. The final osmolality of the lymph-like fluid should be between 280-340 mOsm/L.

To make the lymph-like fluid, molecules consisting of the Polypeptides, insulin, ATP and artificial CSF with elevated Mg²⁺ concentration may be manufactured in a ready to use condition. Optionally, artificial CSF with elevated Mg²⁺ concentration may be manufactured in one container, the mixture of polypeptides, insulin and ATP may be assembled in another container.

The lymph-like fluid composition may also contain other nutrients such as vitamins (e.g., D-Calcium Pantothenate, Choline, Folic acid, i-Inositol, Niacinamide, Pyridoxal, Riboflavin, Thiamine, Vitamin B₁₂ etc.), Amino acids (e.g., L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, antioxidant etc.

The lymph-like fluid composition may also contain oxygen carriers such as bisperfluorobutyl ethylene (oxygenated before use), intermediate molecules of glycolysis (e.g., fructose-1,6-biphophate, glyceraldehyde-3-phosphate, 1,3 bisphosphoglycerate, 3-phosphoglycerate, 2-phosphoglycerateare, phosphoenolpyruvate, pyruvate), enzymes for glycolysis (e.g., hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), fructose-2,6-biphosphate, and intermediates of Krebs cycle.

Although the lymph-like fluid composition is for replacing saline as an irrigating solution during neurosurgical procedures, application of the lymph-like fluid composition before and after surgical procedures may result in even better outcome of the neurosurgical procedures. The irrigating solution may be cooled between 4 to 37° C. before use. The irrigating solution can be removed from the subarachnoid space if necessary following patient's recovery.

Since all surgical procedures followed by tissue edema, the use of this irrigating solution may be extended to all surgical procedures to replace the use of saline as an irrigant.

### EXAMPLE ONE

Making of the Irrigating Solution

Artificial CSF (slightly higher concentration of Mg²⁺) used in this example was made according to table 1.

TABLE 1

| Component | Amount |
|---|---|
| NaCl | 8.182 gram |
| KCl | 0.224 gram |
| CaCl₂.2H₂O | 0.206 gram |
| Na₂HPO₄ | 0.113 gram |
| NaH₂PO₄ | 0.023 gram |
| MgSO₄ | 0.361 gram |
| Glucose | 0.6 gram |
| Sterile water for dilution to 1000 ml | |

Mixture of Sericin peptide (molecular weight 2500-6000 Daltons), Insulin and ATP was made according to table 2.

TABLE 2

| | | |
|---|---|---|
| Sericin peptide | 40 | gram |
| Insulin | 3,000 | μU |
| ATP disodium | 0.55 | milligram |
| Mix these substances in one container | | |

Dissolve the mixture of Sericin peptide, Insulin and ATP in 1000 ml of artificial CSF. Final pH of the composition was adjusted to between 6.8 to 7.0.

### EXAMPLE TWO

Making of the Irrigating Solution

Artificial CSF was made according to table 1 in example one.

Mixture of Gelatin (molecular weight between 20,000-25,000 Daltons), Insulin and ATP was made according to table 3.

TABLE 3

| | | |
|---|---|---|
| Gelatin | 35 | gram |
| Insulin | 3,000 | μU |
| ATP disodium | 0.55 | milligram |
| Mix these substances in one container | | |

Dissolve the mixture of Gelatin, Insulin and ATP in 1000 ml of artificial CSF. Final pH of was adjusted between to 6.8 to 7.0.

### EXAMPLE THREE

Treatment for Brain Edema

The cerebral edema was induced in 12 rats weighing between 250-300 gram. Group one: treatment with saline (6 rats). Group two: treatment with the irrigating solution made according to example one (6 rats). Group three: treatment with the irrigating solution made according to example two (6 rats).

Ketamine/xylazine 30 mg/kg ip was given for anesthesia. A silicone catheter (0.025 OD, 0.012 ID inch) was surgically implanted in the cisterna magna as a draining route. A hole of 3 mm in diameter was drilled on the left side of skull (3 mm lateral to midline and 3 mm in front of the bregma), dura was punctured, an infusing silicone catheter (0.025 OD, 0.012 ID inch) was placed and fixed with glue in the hole into the subarachnoid spaces on the surface of the forebrain.

Focal cerebral edema was produced by middle cerebral artery occlusion. A midline incision on the neck was made. The left common carotid artery, the external carotid artery (ECA) and the internal carotid artery (ICA) were exposed. The ECA was ligated and severed. A 3.0 nylon suture was advanced from the ECA to ICA to block the origin of left middle cerebral artery. The nylon suture was left in place for 3 hours to produce focal cerebral ischemia on left hemisphere supplied by middle cerebral artery. Then the nylon suture was removed to resume blood supply to the ischemia challenged brain for 21 hours.

For group one, at 15 minutes after ischemia, the CSF was removed as completely as possible (usually 0.01-0.02 ml CSF could be withdrawn). After the CSF removal, 3 ml of the 0.9% sodium chloride was continuously infused from the catheter on the left forebrain and was drained out from the catheter in cistema magna. The infusion lasted for 3 hours at a rate of 1 ml/hour.

For group two, at 15 minutes after ischemia, the CSF was removed as completely as possible (usually 0.01-0.02 ml CSF could be withdrawn). After the CSF removal, 3 ml of the irrigating solution made according to example one was continuously infused from the catheter on the left forebrain and was drained out from the catheter in cistema magna. The infusion lasted for 3 hours at a rate of 1 ml/hour.

For group three, at 15 minutes after ischemia, the CSF was removed as completely as possible (usually 0.01-0.02 ml CSF could be withdrawn). After the CSF removal, 3 ml of the irrigating solution made according to example two was continuously infused from the catheter on the left forebrain and was drained out from the catheter in cisterna magna. The infusion lasted for 3 hours at a rate of 1 ml/hour.

Cerebral edema measurement: at 24 hours after cerebral ischemia, all rats were euthanized. Brains were harvested, and two hemispheres were divided along the midline. The wet weight of the hemispheres was measured. The hemispheres were incubated in an oven at 100° C. for 24 hours to obtain the dry weight. Water content was expressed as percentage of wet weight. The formula for calculation was as follows: (wet weight-dry weight)/(wet wight)×100.

Results: in group one, the water content was 88.0±0.5% in ischemic hemisphere and 80.9±0.8% in normal hemisphere. In group two, the water content was 69.7±0.6% in ischemic hemisphere and 69.1±0.4% in normal hemisphere. In group three, the water content was 71.3.0±0.6% in ischemic hemisphere 71.2±0.8% in normal hemisphere. It was concluded that irrigating solution made according to example one and irrigating solution made according to example two significantly reduced brain edema (P<0.01 unpaired t test).

While my above description contains many specifics, these should not be construed as limitations on the scope of the invention, but rather as illustrative examples.

1. An irrigating solution for neurosurgical procedures comprising an artificial cerebrospinal fluid and at least one component selected from the following: the Polypeptides (molecules primarily consisting of chemically-linked amino acids), insulin, ATP.

2. An irrigating solution for surgical procedures comprising an artificial cerebrospinal fluid (Na 120-155 meq/L, K 0.1-5.0 meq/L, Ca 0.1-3.0 meq/L, P 0.1-10 meq/L, Cl 120-155 meq/L, Mg 0.4-5 meq/L, Glucose 0-240 mg/dl and water).

3. An irrigating solution for neurosurgical procedures, as claimed in claim 1, wherein said artificial cerebrospinal fluid comprising: Na 120-155 meq/L, K 0.1-5.0 meq/L, Ca 0.1-3.0 meq/L, P 0.1-10 meq/L, Cl 120-155 meq/L, Mg 0.4-5 meq/L, Glucose 0-240 mg/dl and water.

4. An irrigating solution for neurosurgical procedures, as claimed in claim 1, wherein said polypeptides are present in concentration of about 0.1-30 gram per 100 ml.

5. An irrigating solution for neurosurgical procedures, as claimed in claim 4, wherein said polypeptides are sericin polypeptides.

6. An irrigating solution for neurosurgical procedures, as claimed in claim 4, wherein said polypeptides are gelatins.

7. An irrigating solution for neurosurgical procedures, as claimed in claim 4, wherein said polypeptides are albumin.

8. An irrigating solution for neurosurgical procedures, as claimed in claim 1, wherein said insulin is present in a concentration of about 0.01 to 1000 μU/ml.

9. An irrigating solution for neurosurgical procedures, as claimed in claim 1, wherein said ATP is present in a concentration of about 0.0001 mM to 0.05 mM.

10. An irrigating solution for neurosurgical procedures according to claim 1, further comprises at least one component selected from the group consisting of: Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, oxygen carriers (such as bis-perfluorobutyl ethylene and oxygenated before use), intermediates of glycolysis (such as fructose- 1,6-biphophate, glyceraldehyde-3-phosphate, 1,3 bisphosphoglycerate, 3-phosphoglycerate, 2-phosphoglycerateare, phosphoenolpyruvate, pyruvate, lactate), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (acetoacetate, β-hydroxybatyrate), intermediates of krebs cycle, calcium channel blockers, calcium chelators, Sodium channel blockers, potassium channel blockers, potassium channel openers, free radical scavengers—Antioxidants, GABA agonists, GABA receptor antagonists, polyamine site antagonists, Glycine site antagonists, protein kinase inhibitors, Serotonin agonists, Nitric oxide inhibitors, opiod antagonists, glutamate antagonists, AMPA antagonists, adenosine receptor antagonists, Kainate antagonist, NMDA antagonists (such as CGS 19755, Nimodipine, DP-b99 and Flunarizine, Aptiganel, CP-101,606, Dextrorphan, destromethorphan, metamine, MK-801, NPS 1506, GYKI 52466, NBQX, YM90K, YN872, ZK-200775, MPQX, SYM 2081, Bay x 3072, Remacemide, ACEA 1021, GV 150026, Clomethiazole, Eliprodil, Ifenprodil, Lubeluzole, Naloxone, Nalmefenem, Citicoline, Fosphenytoin, Lubeluzole, 619C89, BMS-204352), Growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells( such as co-enzyme A, co-enzyme Q, or cytochrome C), agents known to reduce cellular demand for energy (such as phenytoin, barbital, or lithium).

11. An irrigating solution for neurosurgical procedures according to claim 1, the final pH value is about 6.8 to 7.35.

12. An irrigating solution for surgical procedures comprising an artificial cerebrospinal fluid and at least one component selected from the following: the Polypeptides (molecules primarily consisting of chemically-linked amino acids), insulin, ATP.

13. An irrigating solution for surgical procedures, as claimed in claim 12, wherein said polypeptides are present in concentration of about 0.1-30 gram per 100 ml.

14. An irrigating solution for surgical procedures, as claimed in claim 13, wherein said polypeptides are sericin polypeptides.

15. An irrigating solution for surgical procedures, as claimed in claim 13, wherein said polypeptides are gelatins.

16. An irrigating solution for surgical procedures, as claimed in claim 13, wherein said polypeptides are albumin.

17. An irrigating solution for surgical procedures, as claimed in claim 12, wherein said insulin is present in a concentration of about 0.01 to 1000 μU/ml.

18. An irrigating solution for surgical procedures, as claimed in claim 12, wherein said ATP is present in a concentration of about 0.0001 mM to 0.05 mM.

19. An irrigating solution for surgical procedures, as claimed in claim 12, wherein said artificial cerebrospinal fluid comprising: Na 120-155 meq/L, K 0.1-5.0 meq/L, Ca 0.1-3.0 meq/L, P 0.1-10 meq/L, Cl 120-155 meq/L, Mg 0.4-5 meq/L, Glucose 0-240 mg/dl and water.

20. An irrigating solution for neurosurgical procedures, as claimed in claim 1, wherein said Polypeptides comprising mainly chemically-linked amino acids with molecular weight between 1000 to 30,000 Daltons.

21. An irrigating solution for surgical procedures, as claimed in claim 12, wherein said Polypeptides comprising mainly chemically-linked amino acids with molecular weight between 1000 to 30,000 Daltons.
